SEQUENCE LISTING (11 sequences)

SEQ ID NO: 1 - RNA, 20 nt, Artificial Sequence
  Description of Artificial Sequence Synthetic oligonucleotide
uagcaccauuugaaaucagu

SEQ ID NO: 2 - DNA, 20 bp, Artificial Sequence
  Description of Artificial Sequence Synthetic oligonucleotide
acgcactcagctagtgacca

SEQ ID NO: 3 - RNA, 19 nt, Artificial Sequence
  Description of Artificial Sequence Synthetic oligonucleotide
uagcaccauuugaaaucag

SEQ ID NO: 4 - RNA, 18 nt, Artificial Sequence
  Description of Artificial Sequence Synthetic oligonucleotide
uagcaccauuugaaauca

SEQ ID NO: 5 - RNA, 17 nt, Artificial Sequence
  Description of Artificial Sequence Synthetic oligonucleotide
uagcaccauuugaaauc

SEQ ID NO: 6 - DNA, 29 bp, Artificial Sequence
  Description of Artificial Sequence Synthetic probe
taactagtgactgatttcaaatggtgcta

SEQ ID NO: 7 - DNA, 9 bp, Artificial Sequence
  Description of Artificial Sequence Synthetic probe
cactagtta

SEQ ID NO: 8 - RNA, 23 nt, Artificial Sequence
  Description of Artificial Sequence Synthetic oligonucleotide
uagcaccauuugaaaucaguguu

SEQ ID NO: 9 - RNA, 20 nt, Artificial Sequence
  Description of Artificial Sequence Synthetic oligonucleotide
uagcaccauuugaaaucagu

SEQ ID NO: 10 - RNA, 22 nt, Artificial Sequence
  Description of Artificial Sequence Synthetic oligonucleotide
cguuagccguaccguugaaucc

SEQ ID NO: 11 - RNA, 22 nt, Artificial Sequence
  Description of Artificial Sequence Synthetic oligonucleotide
ccccuaucacgauuagcauuaa

(19) United States
(12) Patent Application Publication
(10) Pub. No.: US 2011/0027798 A1
(43) Pub. Date: Feb. 3, 2011

(54) Hybridization Quantitation Method for Modified Micro-RNA and -DNA Based Oligonucleotides

(75) Inventors: Kenneth K. Chan, Dublin, OH (US); Zhongfa Liu, Worthington, OH (US); Zhiliang Xie, Columbus, OH (US); Guido Marcucci, Powell, OH (US); John C. Byrd, Columbus, OH (US); Natarajan Muthusamy, Galloway, OH (US); Ramiro Garzon, Columbus, OH (US); Shujun Lui, Dublin, OH (US)

Correspondence Address: MACMILLAN SOBANSKI & TODD, LLC, ONE MARITIME PLAZA FIFTH FLOOR, 720 WATER STREET, TOLEDO, OH, 43604-1619, US

(73) Assignee: THE OHIO STATE UNIVERSITY RESEARCH FOUNDATION, Columbus, OH (US)

(21) Appl. No.: 12/935,775

(22) PCT Filed: Mar. 31, 2009
(86) PCT No.: PCT/US2009/038863

§ 371 Date: Oct. 21, 2010

Related U.S. Application Data
(60) Provisional application No. 61/072,599, filed on Mar. 31, 2008.

(51) Int. Cl. C12Q 1/68; C07H 21/04

(52) U.S. Cl. 435/6; 536/23.1; 536/24.3; 536/24.5

(57) ABSTRACT

Described herein is a method for the qualitative and/or quantitative determination of an analyte in a test sample which includes base pairing at least one oligonucleotide to a capture template having an overhang; and, hybridizing with a detection probe.

### CROSS-REFERENCE TO RELATED APPLICATIONS

This application claims the benefit of U.S. Provisional Application No. 61/072,599, filed Mar. 31, 2008, the disclosure of which is expressly incorporated herein by reference.

### STATEMENT REGARDING FEDERALLY SPONSORED RESEARCH

This invention was made with not made with any Government support and the Government has no rights in this invention.

### TECHNICAL FIELD AND INDUSTRIAL APPLICABILITY OF THE INVENTION

Described herein is a method for the qualitative or quantitative determination of an analyte in a test sample which includes base pairing a nucleotide to a capture template having an overhang; and, hybridizing with a detection probe.

### BACKGROUND OF THE INVENTION

MicroRNAs (miRNAs) are small non-coding RNAs that bind to target microRNAs and regulate their expression. Recent evidence has suggested the microRNAs involvement in chronic lymphocytic leukemia (CLL) transformation and non-small-cell lung cancer (NSCLC).

Naturally occurring microRNAs (miRNAs) are 19- to 25-nucleotide (nt) transcripts cleaved from 70 to 100 nt hairpin primary precursors, which are encoded in the genomes of invertebrates, vertebrates and plants. Although, the biological functions of miRNAs remain to be fully understood, for the most part, these non-coding RNAs seem to regulate protein expression by either causing degradation or translation inhibition of the corresponding coding mRNA. Recent work indicates that miRNAs are involved in human tumorigenesis. Following earlier reports of miRNA de-regulation in solid tumors and hematological malignancies, elegant studies have now shown that a subset of miRNAs may act as oncogenes or tumor suppressors. For instance, the miR-17-92 clusters, is up-regulated in diffuse B-cell lymphomas, lung and stomach cancer. The ectopic expression of this miRNA induces proliferation, decreases apoptosis and cooperates with C-Myc to develop lymphoma in mice. Other miRNAs, when down-regulated, instead allow re-expression of oncogenes that contribute to the malignant transformation, and/or more aggressive phenotypes. For example, it has been reported that miR-15a and miR-16-1, which are deleted or down-regulated in approximately 60% of B cell CLL, targets the antiapoptotic gene BCL-2. Similarly, the miR-29 family of miRNAs is down-regulated in aggressive non-small cell lung cancer (NSCLC) and chronic lymphocytic leukemia (CLL). Further studies have shown that these miRNAs target the oncogenes Tc1-1, Mc1-1 and DNMT3A and 3B. Indeed, restoring miR-29 expression induces apoptosis and hampers tumorigenesis in a xenograft model of lung cancer. Collectively, these data indicates that miRNAs might be potential therapeutic targets for synthetic RNA oligonucleotides that function as antagomiRNAs or miRNAs.

However, there is a need to move these synthetic compounds forward to the clinic. Also, a sensitive and specific analytical tool is needed for measurement of the drug levels in circulation and tissues, thereby allowing of pharmacokinetic characterization and tissue and intracellular distribution.

Due to low levels of these compounds, normal analytical methodologies, such as HPLC and even capillary electrophoresis methods, are not likely to have adequate sensitivity. Several groups have described microarray methods for monitoring miRNA expression and these efforts have provided a qualitative overview of miRNA expression patterns in cell lines and in normal and diseased human tissues. Although gene expression microarrays and quantitative transcript-specific PCR assays have proven to be powerful tools for validating initial observations of their biological activities and their underlining mechanisms of endogenous miRNAs, none of these methods has been applied to characterize the levels of synthetic miRNAs.

### SUMMARY OF THE INVENTION

In a first broad aspect, there is provided herein a method for the qualitative or quantitative determination of an analyte in a test sample, comprising: base pairing at least one oligonucleotide to a capture template having an overhang; and hybridizing with a detection probe.

In a first broad aspect, there is provided herein a method for the qualitative or quantitative determination of an analyte in a test sample, comprising: a) base pairing at least one oligonucleotide to a capture probe having an overhang; and, b) hybridizing with a detection probe, wherein the detection probe is complementary to the overhang of the capture probe.

In another broad aspect, there is provided herein a method for the qualitative or quantitative determination of an analyte in a test sample, comprising: a) base pairing at least one oligonucleotide to a capture probe having a 5′-overhang; wherein the capture probe comprises: i) an oligonucleotide having a sequence complementary to the target analyte, and ii) a 5′-overhang having a sequence complementary to the sequence of a detection probe; and, b) hybridizing with the detection probe, wherein the detection probe comprises: i) an oligonucleotide having a sequence with substantially no similar match with the sequence of the 5′-overhang of the capture probe, and ii) a marker.

In certain embodiments, the detection probe has substantially no similar match with the sequence of the overhang of the capture probe; and little base pairing occurs between the detection probe and the capture probe.

In certain embodiments, the detection probe includes an oligonucleotide sequence comprising TAA CTA GTG.

In certain embodiments, the capture probe comprises a biotin labeled capture probe.

In certain embodiments, the capture probe includes a 9-mer overhang at 5′ terminal, wherein the 9-mer overhang is complementary to the detection probe.

In certain embodiments, the capture probe comprises SEQ ID NO:6.

In certain embodiments, the detection probe comprises SEQ ID NO:7.

In certain embodiments, the capture probe comprises SEQ ID NO:6, and the detection probe comprises SEQ ID NO:7.

In certain embodiments, the oligonucleotide comprises one or more of: modified miRs; synthetic miRs; antagomirs; SNP of miRs; siRNAs, and modified or non-modified DNA based oligonucleotides.

In certain embodiments, the oligonucleotide comprises SEQ ID NO:1.

In certain embodiments, the oligonucleotide comprises SEQ ID NO:8.

In certain embodiments, the oligonucleotide comprises SEQ ID NO:9.

In certain embodiments, the oligonucleotide comprises SEQ ID NO:10.

In certain embodiments, the oligonucleotide comprises SEQ ID NO:11.

In certain embodiments the method includes detecting the detection probe using fluorescence.

In another broad aspect, there is provided herein a capture probe comprising: i) an oligonucleotide having a sequence complementary to a target analyte, and ii) a 5′-overhang having a sequence complementary to the sequence of a detection probe.

In another broad aspect, there is provided herein a capture probe comprising SEQ ID NO: 5.

In another broad aspect, there is provided herein a detection probe comprising: i) an oligonucleotide having a sequence with substantially no similar match with the sequence of the 5′-overhang of a capture probe, and having little base pairing between the detection probe and the capture probe; and ii) a marker. In certain embodiments, the oligonucleotide sequence comprises TAA CTA GTG.

In certain embodiments, the marker comprises a hapten.

In certain embodiments, the marker comprises an immunohistochemical marker.

In certain embodiments, the marker comprises digoxigenin.

In certain embodiments, the detection probe comprises an alkaline phosphatase-conjugated antidigoxigenin antibody.

In another broad aspect, there is provided herein a detection probe comprising SEQ ID NO: 6.

In another broad aspect, there is provided herein a method for assessing pharmacologically specific effects of one or more synthetic microRNAs, comprising the step of: using the method described herein.

In another broad aspect, there is provided herein a method for characterizing intracellular pharmacokinetics and/or and pharmacodynamics data of a modified microRNA and its preclinical pharmacokinetics, comprising the step of: using the method described herein.

In another broad aspect, there is provided herein a method for determining 3′-cholesterol or other moiety block microRNAs or antagomirs, comprising the step of: using the method described herein.

In another broad aspect, there is provided herein a method for qualitative and/or quantitative determination of an analyte in a test sample, comprising the step of: using the method described herein.

In another broad aspect, there is provided herein a method for detecting a microRNA at a sensitivity of at least about 30 pM, comprising the step of: using the method described herein.

In another broad aspect, there is provided herein a method for determining one or more of structural confirmation, identification and differentiation with metabolites or other endogenous substances, comprising the step of: using the method described herein.

In another broad aspect, there is provided herein a method for providing relative concentrations of species that are measurable in low concentration ranges, comprising the step of: using the method described herein.

In another broad aspect, there is provided herein a method for the qualitative or quantitative determination of an analyte in a test sample, comprising: mixing a capture template solution and a quantity of an oligonucleotide; incubating the mixture for a sufficient time for hybridization to occur; transferring the mixture to a biotin-binding protein coated well plate; incubating to allow attachment of a biotin-labeled capture template to the coated wells; adding a ligation solution containing a ligase and detection probe; incubating, then washing to remove any unligated detection probe; adding an endonuclease reagent; incubating to cleave any truncated duplex present, then washing and blocking; adding a detection probe, incubating, then washing, adding a substrate solution containing a fluorescent substrate, incubating; and, measuring fluorescence intensity.

In another broad aspect, there is provided herein a kit, comprising one or more reagents for detecting one or more miRs in a test sample from a subject, as compared to a control, wherein the reagents comprise a capture probe having a overhang, and a detection probe complementary to the overhang of the capture probe.

In certain embodiments, the kit includes a detection probe that has substantially no similar match with the sequence of the overhang of the capture probe; and little base pairing occurs between the detection probe and the capture probe. In certain embodiments, the kit includes a detection probe that has an oligonucleotide sequence comprising TAA CTA GTG.

In certain embodiments, the kit includes a capture probe comprising a biotin labeled capture probe.

In certain embodiments, the kit includes a capture probe having a 9-mer overhang at 5′ terminal, wherein the 9-mer overhang is complementary to the detection probe.

In certain embodiments, the kit includes a capture probe comprising SEQ ID NO:6.

In certain embodiments, the kit includes a detection probe comprising SEQ ID NO:7.

In certain embodiments, the kit includes a capture probe comprising SEQ ID NO:6, and a detection probe comprising SEQ ID NO:7.

In certain embodiments, the kit includes a detection probe comprising a digoxigenin detection probe complementary to the overhang of the capture probe.

In certain embodiments, the kit includes a capture probe comprising a biotin labeled capture probe.

In certain embodiments, the kit includes a capture probe comprising: i) an oligonucleotide having a sequence complementary to a target analyte, and ii) an overhang having a sequence complementary to the sequence of a detection probe; and, wherein the detection probe comprises: i) an oligonucleotide having a sequence with substantially no similar match with the sequence of the overhang of the capture probe, and ii) a marker.

Various objects and advantages of this invention will become apparent to those skilled in the art from the following detailed description of the preferred embodiment, when read in light of the accompanying drawings.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the United States Patent and Trademark Office upon request and payment of the necessary fee.

FIG. 1: A schematic illustration of a scheme of hybridization-ligation ELISA.

FIGS. 2A-2D: Calibration curves of 2-MeOPSmiR29b in 10% mouse plasma (FIGS. 2A and 2B) and in K562 cell lysate (FIGS. 2C and 2D).

FIG. 3. The stability profiles of 2-MeOPSmiR29b in EDTA-treated mouse plasma at different temperatures.

FIG. 4: Cross-reactivity of putative metabolites (5′-N-1,3′-N-1,3′-N-2, and 3′-N-3), and scrambled 2-MeOPSmiR29b with 2-MeOPSmiR29b. The small insert showed the cross-reactivity at low concentrations.

FIG. 5: Plasma concentration-time profile of 2-MeOPSmiR29b in mice following an intravenous bolus dose at 7.5 mg/kg. Squares represent mean measured concentrations, upper and lower bars represent SD with n=6, and line represent the fitted curve to a two-compartment model with instantaneous input.

FIGS. 6A-6B: Intracellular concentration-time profiles of 2-MeOPSmiR29b in peripheral blood cells (FIG. 6AA) and bone marrow (FIG. 6B) in mice following an intravenous bolus dose at 7.5 mg/kg.

FIG. 7—Table 1. Within-run and between-run validation parameters of 2-MeOPSmiR29b in 10% mouse plasma (all n=6).

FIG. 8: Table 2. Within-run and between-run validation parameters of 2-MeOPSmiR29b in K562 cell lysates (all n=6).

FIG. 9: Table 3. Relevant Pharmacokinetic Parameters of 2-O-Me-miRNA29b in mice following i.v. dosing at 7.5 mg/kg.

FIG. 10: Table 4. Oligonucleotides, sequences and SEQ ID Nos.

### DETAILED DESCRIPTION OF THE PREFERRED EMBODIMENT

Throughout this application, various publications are referred to by citations within parentheses and in the bibliographic description, immediately preceding the claims. The disclosures of these publications are hereby incorporated by reference into the present disclosure to more fully describe the state of the art to which this invention pertains.

Assay generally includes a method of screening for a desired substance, and generally includes a method for the qualitative or quantitative determination of an analyte in a test sample.

In a broad aspect, there is described herein is an assay that is based on a two-step hybridization technique, with synthetic microRNAs binding to a biotin labeled 9-mer longer capture probe followed by ligation with a 9-mer digoxigenin (Dig) detection probe complementary to the 9-mer overhang of the capture probe. The Dig was detected by anti-Dig-alkaline phosphatase system using fluorescence.

In a broad aspect, there is provided herein an ultrasensitive hybridization ELISA assay for synthetic microRNAs including miR29b, miR-16 and antagomiR-155 in biological matrices was developed and validated.

In a particular aspect, there is provided herein a system which uses a highly sensitive and specific complementary ELISA and a mass spectrometric method for measurement of modified microRNAs.

In another aspect, the system is useful to measure synthetic small interfering RNA (siRNA) and their analogs using the same 9-mer digoxigenin (Dig) detection probe, complementary to the 9-mer overhang of the biotin labeled capture probe, which has the sequence complementary to that of siRNAs of either strand. Interfering RNAs are small double strand RNAs that help to control gene functions that relate to normal and pathological states.

This provides a distinct advantage, since currently there is no method to determination of 3′-cholesterol or other moiety block microRNAs or antagomiRNAs. In contrast, the one-step hybridization ELISA method described herein can detect 3′-cholesterol or other moiety block microRNA with high sensitivity of 30 pM, which is the sole effective tool to be used for characterization of intracellular pharmacokinetics and trafficking.

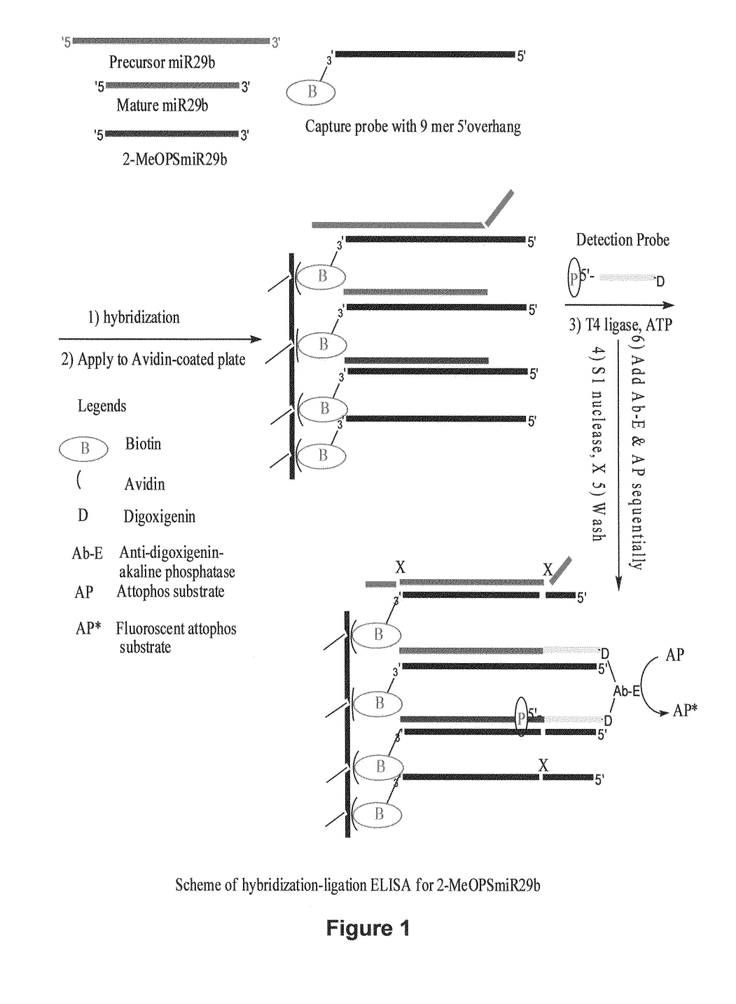

The inventors developed a hybridization-ligation ELISA method for GTI-2040 and G3139 to quantify 2-MeOPSmiR29b, as schematically shown in FIG. 1 and the protocol for the method as described herein.

While not wishing to be bound by theory, the inventors herein believe that the ELISA method may not differentiate exogenous and endogenous microRNAs, which is the same for TaqMan® MicroRNA Assays (Applied Biosystems, Foster City, Calif.). The inventors herein have developed a LC-MS/MS to measure the modified microRNA with a limit of detection of 5 nM as shown in the case of 2-methoxy-miR155.

The ELISA method described herein can also be useful for structural confirmation, identification and differentiation with metabolites or other endogenous substances such as SNP of microRNAs as demonstrated in the two antisense drugs, G3139 and GTI2040.

The ELISA method described herein can also provide relative concentrations of the species that are measurable at a higher concentration range.

In addition, the same strategy was also adaptable to measure synthetic 2-methoxy phosphothiolate microRNA 16-1 (2-MeOPSmiR16-1) and 2-methoxy phosphothiolate antagomR-155 (2-MeOPSantagomiR155) using the same 9-mer digoxigenin (Dig) detection probe complementary to the 9-mer overhang of the biotin labeled 9-mer longer capture probe with other sequence complementary to that of 2-MeOPSmiR16-1 and 2-MeOPSantagomiR155, respectively.

Human miR-29b, in contrast to other studied animal microRNAs, has been found predominantly localized to the nucleus and substantially accumulated in mitotic HeLa cells. The potential targets of miR29b are: 1) the antiapoptotic protein Mc1-1, which is a member of the Bc1-2 family and its over-expression or mutations have been shown to predict aggressive phenotype in hematologic malignancies, including CLL and AML54a,s4b,54c; 2) DNMTs, the regulators of DNA methylation pattern and now have been identified as a major therapeutic target for epigenetic therapy in hematologic malignancies, including CLL and AMLs4a,s4b,s4c; and, 3) the dihydrolipoamide branched chain acyltransferase component of BCKD, a major enzyme of the metabolic pathway of amino acid catabolism in mammals.

In addition, as Mc1-1 protein has a relatively brief half-life (˜30 min) 54d, it represents an optimal readout for validation of microRNA targeting strategies.

Thus, the validated ELISA method described herein provides the first useful tool to assess pharmacologically specific effects of the synthetic microRNA compounds (e.g., miR29b) timely (within 2-6 hours), before non-specific drug-related mechanisms of apoptosis intervened.

Described herein is a validated non-radioactive hybridization-ligation ELISA method for determination of miRs in a variety of biological matrices. Several of the many advantages of this assay, include, but are not limited to: (a) ultra-sensitive with a lower limit of quantitation (LLOQ) of 5 pM, (b) highly selective toward 3′-end deletion metabolites, (c) negligible matrix effect, (d) simple sample preparation, and (e) can be used in high throughput applications.

This assay is also useful to measure plasma and intracellular levels of synthetic miRs.

In a particular embodiment, described herein is a method for the in vivo characterization of the pharmacokinetics of synthetic microRNAs. For example, a favorable pharmacokinetics of 2-MeOPSmiR29b was observed for its therapeutic application with attainable plasma and intracellular concentration of 2-MeOPSmiR29b and comparable terminal half life of currently clinical-used anti-sense drugs.

This assay is useful with different appropriate capture and detection probes. Further, this assay can be universally applicable in quantification of exogenous synthetic microRNAs and antagomirs in a variety of biological matrices.

The one-step hybridization ELISA method for anti-sense oligomers can also be used to measure 3′-blocked (e.g., cholesterol) synthetic microRNAs and antagomirs.

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. All publications, including patents and non-patent literature, referred to in this specification are expressly incorporated by reference herein.

### Example

### Materials and Methods

Oligonucleotides and Reagents

The oligos and their sequences used were purchased from Dharmacon Inc. (Lafayette, Colo.), while the capture and detection probe were custom synthesized and acquired through Integrated DNA Technologies (Coralville, Iowa).

The capture probe for 2-MeOPSmiR29b used in the two-step hybridization ELISA was designed as a 29mer DNA oligonucleotide with the first 20mer sequence from the 3′-end complementary to 2-MeOPSmiR29b and the 3′-end was attached to a NeutrAvidin-coated 96-well plate via biotin. The 9mer overhang (5′-TAA CTA GTG-3′) serves as a template for the detection probe. A 9-mer DNA phosphorothioate with digoxigenin at the 3′-end and sequence complement to the 5′-end 9mer overhang of the capture probes for 2-MeOPSmiR29b is used as an appropriate detection probe following the hybridization ligation reaction. The purity and identity of each oligomers were verified by HPLC-UV/Mass spectrometry (Ion trap mass spectrometer Model: LCQ, Finnigan, San Jose, Calif.).

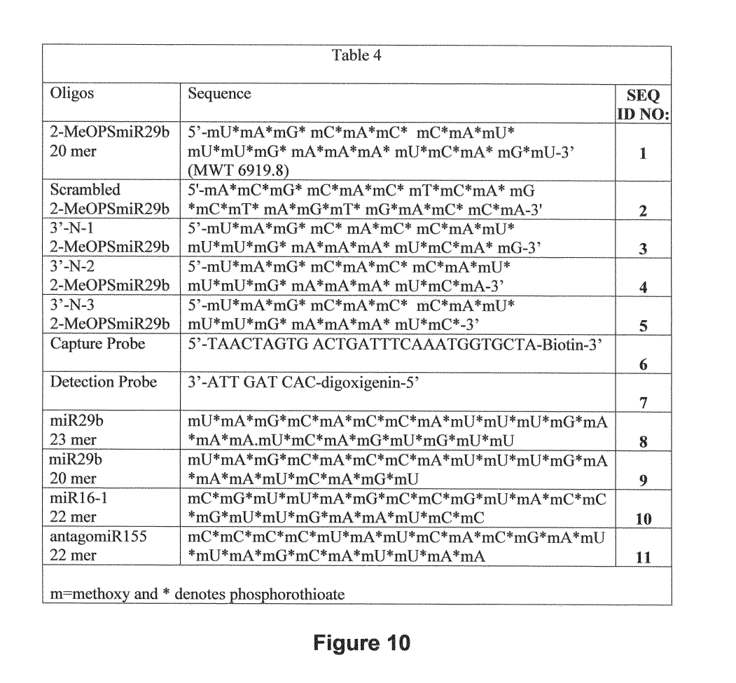

See FIG. 10—Table 4 which shows the sequences for oligonucleotides, capture probes and detection probes.

Reacti-Bind NeutrAvidin-coated polystyrene plates were purchased from Pierce (Rockford, Ill.). 2-MeOPSmiR29b standards were diluted in TE (Tris-HCl and EDTA) buffer containing 10 mM Tris-HCl and 1 mM EDTA (pH=8.0). The hybridization buffer used in preparation of capture probe solution contained 60 mM sodium phosphate, pH 7.4, 1.0 M NaCl, 5 mM EDTA and 0.2% Tween 20. The ligation buffer was prepared as a mixture of 66 mM Tris-HCl, pH 7.6, 10 mM MgCl₂, 10 mM DTT, 1 mM ATP, 5 U/mL T4 DNA ligase and 100 nM detection probe oligonucleotide. T4 DNA ligase and ATP were purchased from Amersham Biosciences (Piscataway, N.J.). The washing buffer used throughout the assay contained 25 mM Tris-HCl, pH=7.2, 0.15 M NaCl, and 0.2% Tween 20. The anti-digoxigenin-AP was obtained from Roche (Indianapolis, Ind.). Attophos and its reconstitution solution were purchased from Promega (Madison, Wis.). Blank mouse plasma was obtained from Innovative Research, Inc. (Southfield, Mich.). Detection was accomplished using a Gemini XS plate reader (Molecular Devices, Sunnyvale, Calif.).

Cell Culture and Cell Lysate Preparation

K562 cell lines were cultured at 37° C. in a 5% CO₂ incubator using in RPMI medium (VWR International, Inc., West Chester, Pa.) supplemented with 10% fetal bovine serum (Invitrogen, Carlsbad, Calif.). Harvested cells (10⁶ per aliquot) were lysed with 1 mL lysis buffer (10 mM Tris-HCl, pH 8.0, 0.5 mM EDTA, 1.0% Triton-X-100).

Hybridization ELISA Assay Procedures

This method is based on a two-step hybridization, first by base pairing 2-MeOPSmiR29b with the capture template with an overhang, followed by hybridization with a detection probe p-5′-TAA CTA GTG-digoxigenin-3′, which is ligated to the analyte. The general procedure of the method is described as follows. Basically, 100 μL of the capture template solution (200 nM) was added to 100 μL 10% mouse plasma or 10% cell lysate diluted with TE buffer containing 2-MeOPSmiR29b, and the solution was mixed in a 96-well raised PCR plate (VWR International, Bridgeport, N.J.). The mixture was incubated at 37° C. for 2.5 h for hybridization. Then 150 μL of the solution was transferred to a NeutrAvidin-coated 96-well plate (Pierce, Rockford, Ill.), which was incubated at 37° C. for 30 min to allow the attachment of biotin labeled capture template to NeutrAvidin-coated wells. The plate was washed six times with washing buffer, and 150 μL ligation solution containing 5 U/mL T4 DNA ligase (USB) and 100 nM detection probe was added to each well. The plate was incubated overnight at 25° C. The plate was washed three times with washing buffer and three times with deionized (DI) water to remove the unligated detection probe. Following addition of 60 U 51 nuclease (per well) solution in 100 mM NaCl, the plate was incubated at 37° C. for 2 hours to cleave the truncated duplex. After washing with deionization (DI) water six times, the plate was blocked with 1:1 Superblock buffer and antibody dilution buffer followed by addition of 150 μL antidigoxigenin-AP diluted with 1:2500 super BSA block buffer (Roche, Indianapolis, Ind.) into each well. The plate was then incubated at room temperature for 0.5 hour with gentle shaking. After washing six times with DI water, 150 μL substrate solution (36 mg Attophos in 60 mL diethanolamine buffer, Promega, Madison, Wis.) was added to each well, and the plate was incubated at 37° C. for 30 minutes. Finally, fluorescence intensity was measured at Ex 430/Em 560 (filter=530 nm) using a Gemini XS fluorescence microtiter plate reader.

Method Validation

The hybridization-ligation ELISA method for 2-MeOPSmiR29b in 10% mouse plasma and 10% K562 cell lysate was validated. Linearity at the concentration range between 5 and 5000 pM was evaluated. Within-day and between-day accuracy and precision were determined at 10 pM (low quality control, QC), 50 pM (medium low, QC), 500 pM (median QC) and 5000 pM (high QC) with 6 replicates in each matrix. Since the drug concentration ranges in animal and cell samples are likely to exceed the upper limit of the calibration curve, extension of the dynamic range was evaluated by dilution. Mouse plasma spiked with 10, 50 and 200 nM 2-MeOPSmiR29b was diluted with 10% mouse plasma to 10, 50 and 200-folds, respectively (n=6). These standards were assayed and the dilution recovery was calculated by the dilution factors.

Stability of 2-MeOPSmiR29b

2-MeOPSmiR29b (1.5 μM) was incubated in EDTA or heparin-pretreated mouse plasma separately at −20, 4, 25, 37° C. and 100 μL aliquots each of these samples were collected at 0, 30 minutes, 1, 2, 4, 8, 24 hours. These aliquots were stored in a −80° C. freezer until analysis. At suitable times, the 2-MeOPSmiR29b concentrations in these samples were determined using the aforementioned ELISA method.

Specificity and Selectivity of the Hybridization-Ligation ELISA

The cross-reactivity of 3′-N-1, N-2, N-3 and 5′-N-1 putative metabolites and control oligomers (scrambled oligonucleotides) of 2-MeOPSmiR29b at the concentration range between 5 μM to 10 nM in 10% mouse plasma in TE buffer was evaluated. The observed fluorescence-concentration profiles of these oligonucleotides were compared with those of the parent 2-MeOPSmiR29b. The cross-reactivity of each metabolite toward 2-MeOPSmiR29b was determined as the percentage of their EC₅₀ values to that of 2-MeOPSmiR29b. The EC₅₀ values were calculated by the nonlinear regression model in SigmaPlot (SPSS, Chicago, Ill.).

Pharmacokinetics of 2-MeOPSmiR29b in Mice

C57BI/6 mice (˜20 g) (Harlan, Indianapolis, Ind.) were used in this study. All animal procedures were performed according to a protocol in compliance with The Ohio State University Laboratory Animal Resources (ULAR) policies, which adhered to the guideline and “Principles of Laboratory Animal Care by National Institutes of Health. For intravenous bolus administration, approximately 50 μL (adjusted by body weight) 2-MeOPSmiR29b dissolved in sterile normal saline as a 2 mg/mL solution was injected through the tail vein resulting in an intravenous bolus dose of 7.5 mg/kg. The blood was removed by cardiac puncture under CO₂ anesthesia at the time schedule of 0 (pre-dose), 0.08, 0.15, 0.25, 0.5, 1, 2, 4, 7 and 24 hours after dosing and was mixed with 3% (v/v) sodium heparin. The blood samples were centrifuged at 1000 g for 5 min and the supernatant and peripheral blood cell of each were collected and kept at −80° C. until analysis. The bone marrows of these mice were also collected at 2, 4, 7 and 24 hours. These samples were diluted properly based on our previous pharmacokinetics study of anti-sense oligonucleotides and these diluted samples were processed according to the procedures for plasma and cell lysate (23).

The 2-MeOPSmiR29b levels in plasma, in PBC and in bone marrow were measured using the ELISA assay described herein. Plasma and PBC concentration-time data were analyzed by WinNonlin computer software (Pharsight 5.0, Mountain View, Calif.) using appropriate pharmacokinetic models. Protein levels in PBC lysate were determined with BCA assay (Pierce, Rockfold, Ill.) and were used to normalize concentration in PBC (nM/mg protein). The PBC levels of 2-MeOPSmiR29b were converted to concentration (nM) using a conversion factor of 2×10⁶ cells to 1 μL cell volume or 1 μg protein to 0.035 μL cell volume (23).

Results

Method Development and Validation

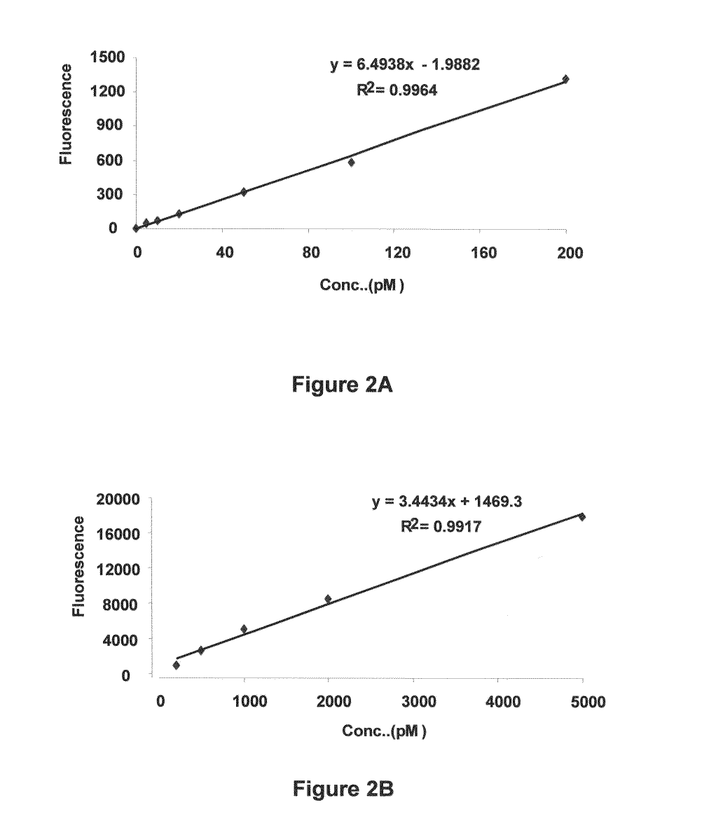

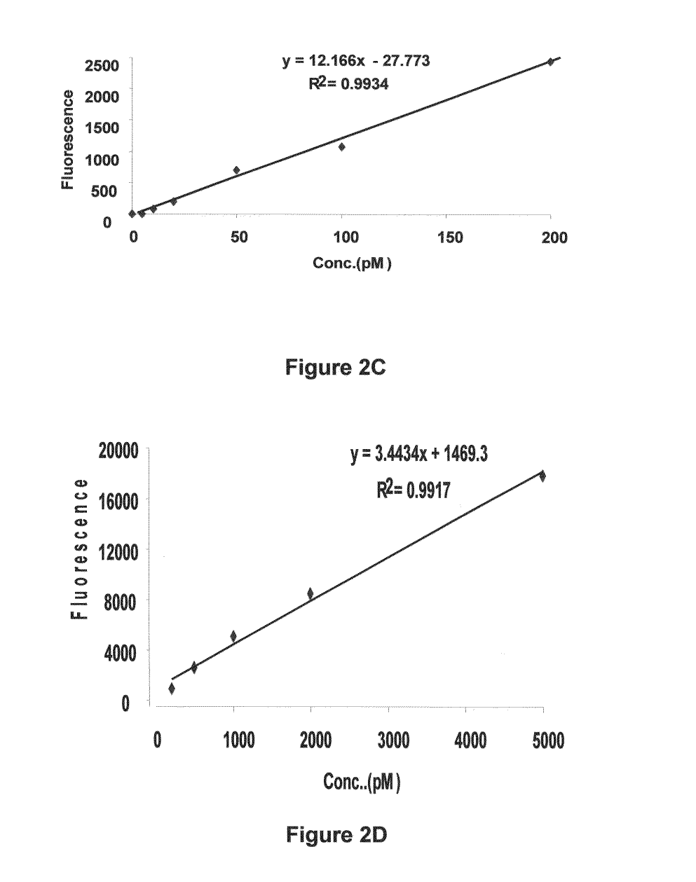

The linearity of this method for quantification of 2-MeOPSmiR29b was initially evaluated in TE buffer (data not shown), and then in 10% mouse plasma (FIGS. 2A and 2B) and 10% K562 cell lysate (FIGS. 2C and 2D). The assay was indeed found to be linear in these matrices in a range between 5 to 5000 pM with a regression coefficient of >0.990. The upper limit for the calibration curve was set at 5000 pM, as above this value the fluorescence response reached a plateau in both plasma and cell lysate. The lowest limit of detection (LLOD) was found to be 5 pM and the lowest limit of quantification (LLOQ) was found to be 10 pM following blank subtraction, respectively.

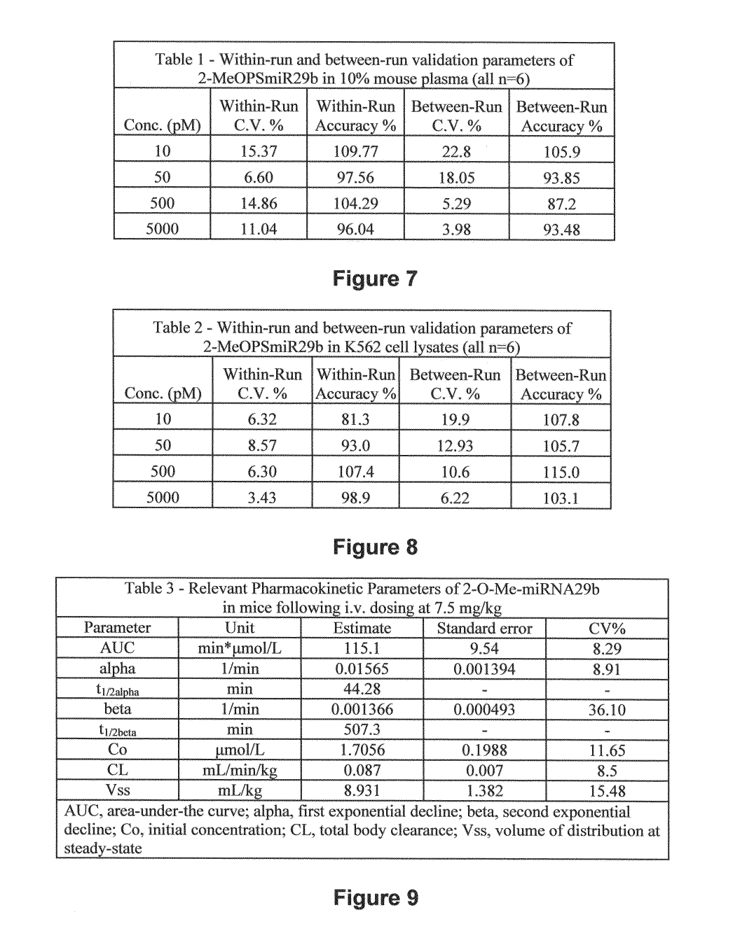

The accuracy and precision of the method was then determined at 10, 50, 500, and 5000 pM (FIG. 7—Table 1 and FIG. 8—Table 2). The within-run accuracy values were 110, 98.6, 104 and 96.0% (n=6) at 10, 50, 500 and 5000 pM, respectively, in 10% mouse plasma (FIG. 7—Table 1), with coefficients of variation (CVs) for each corresponding QCs estimated to be 15.4, 6.60, 14.9, and 11.0%. The between-run accuracy values were 105.9, 93.85, 87.2 and 93.5% at 10, 50, 500 and 5000 pM in 10% mouse plasma, respectively.

Similarly, the within-run accuracy values in 10% K562 cell lysate were found to be 81, 93, 107 and 99% at 10, 50, 500 and 5000 pM, respectively, with CVs for the corresponding QCs estimated to be 6.32, 8.57, 6.30, and 3.43% (FIG. 8—Table 2). The between-run accuracy values were 107.8, 105.7, 115 and 103% at 10, 50, 500 and 5000 pM in 10% cell lysate, respectively.

Specificity

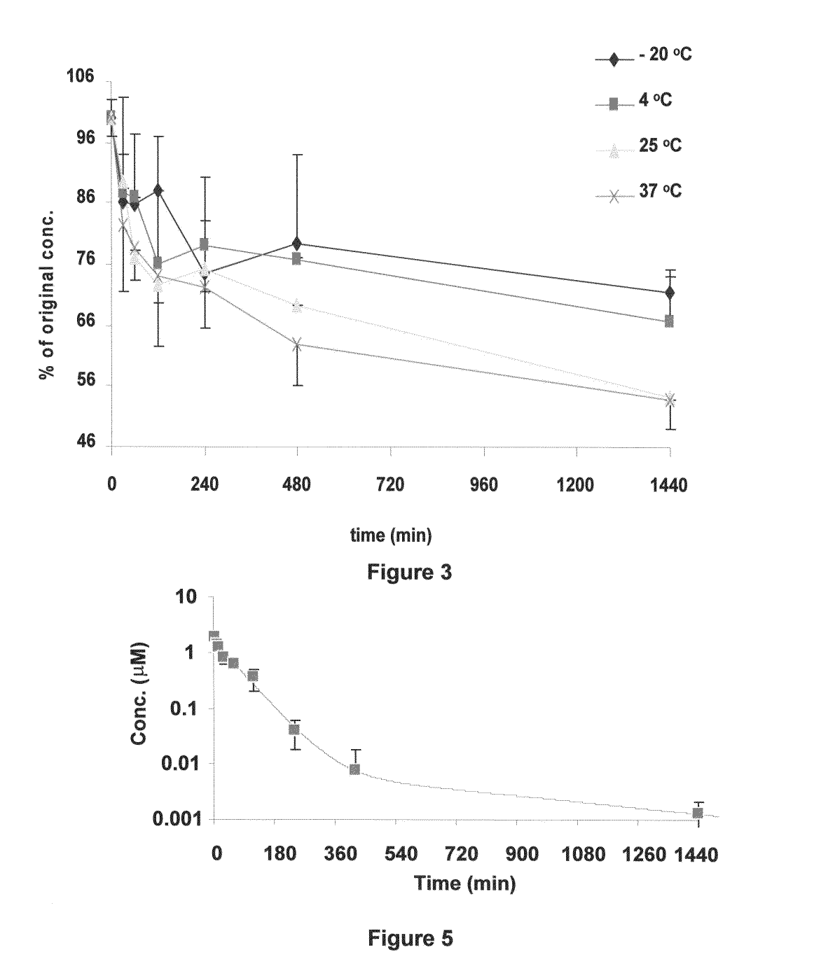

miRNAs are endogenous small RNA existing in precursor and mature forms intracellularly and as circulating nucleic acid in human serum and plasma. The endogenous miRNAs, therefore, represent a source of interference for an analytical system. Therefore, we examined our methods for interferences from endogenous miRNAs and from scrambled oligonucleotides. The fluorescence responses from blank cell matrices and plasma evaluated were found to be negligible and showed no difference from that of the PBS control, indicating a lack of interference from endogenous substances. Furthermore, a scrambled 2-MeOPSmiR oligonucleotide did not seem to compete with the analytes in plasma as shown by their negligible fluorescence signal (FIG. 3), thereby supporting the specificity of the assay described herein.

Cross-Reactivity with Chain-Shortened Metabolites

The hybridization-ligation ELISA method demonstrated its selectivity toward the parent analyte from its putative 3′-N-1 to 3′-N-3 metabolites (FIG. 3). Compared to the concentration-response curve of 2-MeOPSmiR29b, its 3′-N-1 metabolites gave significantly lower fluorescence intensity. The cross-reactivity value for 3′-N-1 2-MeOPSmiR29b was determined to be 2.2% (FIG. 3). No significant cross-reactivity with 3′-N-2 and 3′-N-3 metabolites was observed (FIG. 3). However, the cross-reactivity of the assay with the 5′-N-1 metabolite was about 90%; therefore, this method is considered highly selective but not specific.

Stability of 2-MeOPSmiR29b in Mouse Plasma

The stability of 2-MeOPSmiR29b was evaluated in PBS and in heparin and EDTA pretreated mouse plasma. 2-MeOPSmiR29b was found to be quite stable in PBS for 24 hours (data not shown); however, it decomposed biexponentially in heparinized (not shown in figure) and in EDTA-pretreated mouse plasma in a temperature dependent manner. At 25° C. 2-MeOPSmiR29b in heparinized and EDTA treated plasma degrades with initial half-lives of 12 and 40 minutes, respectively (FIG. 4). The terminal half-lives in both media were about 50 hours at this temperature. About 80, 70, 55 and 50% of 2-MeOPSmiR29b decomposed at 37, 25, 4, and −20° C. in 24 hours in both media.

Pharmacokinetics of 2-MeOPSmiR29b in Mice

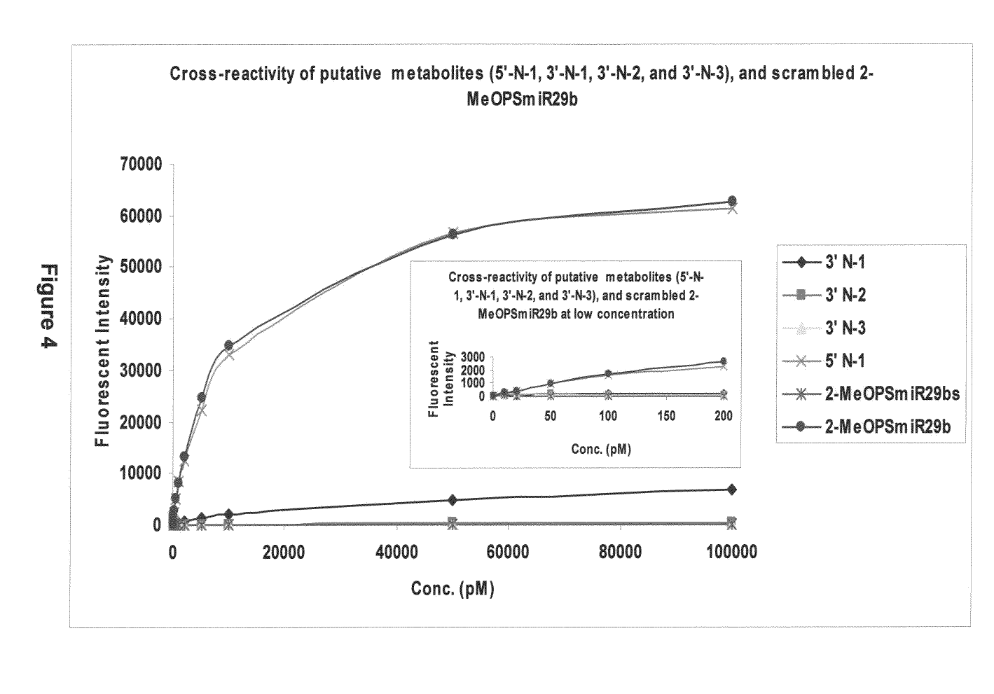

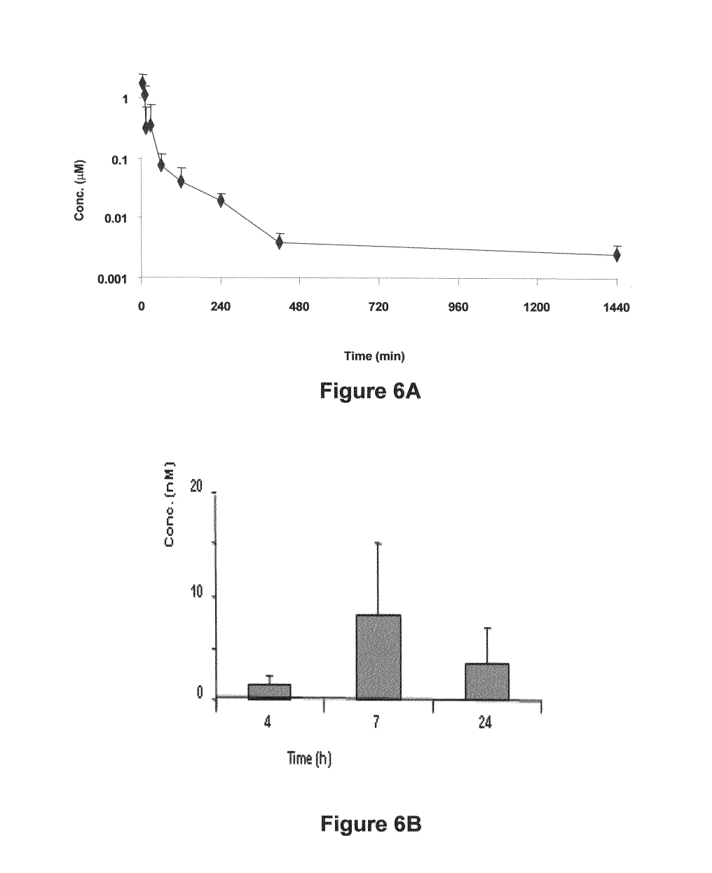

Using the ultra-sensitive ELISA method, the pharmacokinetics of 2-MeOPSmiR29b was investigated in C57BL/6 mice following an i.v bolus dose at 7.5 mg/kg formulated in saline as a 2 mg/mL solution. As shown in FIG. 5, following a 7.5 mg/kg intravenous bolus dose, plasma concentrations of 2-MeOPSmiR29b reached ˜1.9 μM at 5 minutes and declined with time to ˜0.0013 μM at 24 hours post dose. Analysis of the data by a two-compartment model showed that the terminal half life of 8.5 h, AUC 115.1 min*μM, the total body clearance (CL) 0.087 mL/min/kg and the volume of distribution at steady-state 8.9 mL/kg (FIG. 8—Table 3). Interestingly, 2-MeOPSmiR29b levels in peripheral blood cells (PBC) were measurable and reached the Cmax of 1.67 μM at 5 minutes, then declined over time to ˜0.0025 μM at 24 hours (FIG. 6A). Comparison of 2-MeOPSmiR29b plasma and PBC levels indicated that plasma levels were always higher during the first 7 hours after dosing, suggesting the existence of a lower uptake gradient for 2-MeOPSmiR29b in PBC. Importantly and germane to leukemia patients, 2-MeOPSmiR29b was also taken up in bone marrow, where it achieved a concentration of 0.0020 μM at 4 hours, peaked at 0.0072 μM at 7 hours, and then decreased to 0.0036 μM at 24 hours (FIG. 6B). The latter, however, was still higher than the concentration observed in PBC and plasma at the same time point.

Discussion

Currently, several methods, e.g., microarray, northern blot analysis and RT-PCR have been reported for screening endogenous miRNAs. However, none of these methods was used to characterize exogenous miRNAs. Recently, several hybridization-based ELISA methods have been successfully developed for the determination of polynucleotides in biological matrices. However, these ELISA methods have not been applied to quantify endogenous miRNAs and their applicability remains questionable because of the extremely low endogenous miRNA levels.

To explore miRNAs as potential therapeutic agents, modified synthetic miRNAs with the same sequence to those of endogenous miRNAs or modified synthetic antagomiRNAs with the sequence complementary to those of endogenous miRNAs have been prepared. Several reports have demonstrated that interference with exogenous miRNAs or antagomirs may be an effective therapeutic strategy for several diseases in preclinical settings.

However, no pharmacokinetics of exogenous miRNAs has been reported possibly partly due to the lack of proper analytic methods for quantification of exogenous miRNAs. Thus, potential pharmacokinetic correlation with their target modulations and dose optimization remains unknown.

To bridge the gap, herein the inventors developed a two-step method to quantify a modified synthetic 2-MeOPSmiR29b to replace low levels of the endogenous tumor suppressor miRNA29b in leukemia cells. The choice of miRNA29b as proof-of-principle target was due to the potentially important biologic role of this miRNA. Human miRNA-29b have several validated targets: 1) the antiapoptotic protein Mc1-1, which is a member of the Bc1-2 family and its over-expression or mutations have been shown to predict aggressive phenotype in hematologic malignancies, including CLL and AML, 2) DNMTs, the regulators of DNA methylation pattern now have been identified as a major therapeutic target for epigenetic therapy in hematologic malignancies, 3) the dihydrolipoamide branched chain acyltransferase component of branched chain α-ketoacid dehydrogenase (BCKD), a major enzyme of the metabolic pathway of amino acid catabolism in mammals.

Previously, using the hybridization-based ELISA method, we have successfully developed highly sensitive methods for quantification of two antisense oligonucleotides G3139 and GTI-2040. These oligonucleotides are essentially short DNA sequence, while miRNAs are short RNA molecules and synthetic miRNAs are further structurally modified. Whether these modified RNAs could efficiently hybridize with DNA oligonucleotide templates by Watson-Crick base-pairing, as well as the ability of the modified RNAs to enzymatically ligate with a detection probe in the presence of ligase and ATP, was unknown.

Here, we successfully demonstrate that the approach for antisense compounds, including ligation to our custom-designed 9-mer detection probe used for the antisense GTI-2040 is applicable to the assay for 2-MeOPSmiR29b.

In the current assay, we designed a capture probe with its sequence complementary to that of 2-MeOPSmiR29b with a 3′ 9-mer overhang with its sequence complementary to that of 9-mer detection probe and 5′-end digoxigenin (Dig-label). The linearity of the method for 2-MeOPSmiR29b was tested under a protocol similar to that of clinically used DNA oligonucleotide compounds. We have found that 2-MeOPSmiR29b was able to effectively hybridize to the designed capture probe and ligate to the detection probe in the presence of T4 ligase and ATP. These formed well bound duplexes (29-mer duplex for 2-MeOPSmiR29b) which were resistant to the S1 digestion and subsequently detected by the Dig fluorescence system.

While no metabolism study for synthetic miRNAs has been published, the inventors herein now believe that, in vitro cell and in vivo, the 3′-end chain of 2-MeOPSmiR29b may be subjected to 3′-exonucleases similar to antisense compounds to generate chain-shortened metabolites. These potential metabolites may interfere with the quantification of 2-MeOPSmiR29b. Therefore, the cross-activity of these putative metabolites 3′-N-1, 3′-N-2, and 3′-N-3 and 2-MeOPSmiR29b was tested. As shown in FIG. 5, the 3′-N-1 putative metabolite gave only a 2.2% of cross-activity to 2-MeOPSmiR29b, although the 5′-N-1 putative metabolite showed a high (about 90%) cross-activity. It is believed by the inventors herein that this cross reactivity will not pose a significant problem in practice, since 5′-end metabolites may be formed at very low levels.

Following evaluation of the selectivity, the method was validated and the results meet the commonly accepted validation criteria. Notably, the ELISA method for determination of 2-MeOPSmiR29b offers higher sensitivity when compared to those of anti-sense drugs. The LLOD of the method is 5 pM and the LLOQ of the method is 10 pM, 5 fold more sensitive than those of GTI-2040 and G3139. The validated hybridization-ligation ELISA method was also applied to pharmacokinetic study of 2-MeOPSmiR29b in C57BI/6 mice. The ultra-sensitivity of the ELISA assays allows characterization of pharmacokinetics of 2-MeOPSmiR29b for the first time. Specifically, we were able to measure plasma concentration of 2-MeOPSmiR29b up to 24 hours after intravenous bolus dose at 7.5 mg/kg in mice and obtain a mean terminal half-life of about 8 hours. The observed highest mean plasma concentration was nearly 2 μM, which declined to 1.3 nM at 24 hours. Before 6 hours, the concentrations were well above 20 nM, the concentration range of which was previously used for transfection experiments. Others have found that cholangiocarcinoma KMCH cells transfected with 20-50 nM mir-29b precursors showed decreased Mc1-1 reactivity with no change in Bc1-2 activity. Also, others have reported that when HEK293 cells transfected with as little as 0.2 nmole of miR29b achieved BCKD activity in releasing CO₂ for over 24 hours. Still others have transfected A549 and H1299 cells with 50 nM mir29b and found increased expression of Fhit and Wwox proteins. Therefore, the achievable in vivo microRNA concentrations appear to be well above the active concentration range of several microRNAs used in vitro.

The in vivo study with 2-MeOPSmir29b has demonstrated that, at a dose as low as 7.5 mg/kg with no apparent toxicity, micromolar concentrations of the synthetic microRNA in circulation can be achieved. This dose is 10-fold lower than the doses used for other cholesterol-modified synthetic RNAs or antagomirs (>80 mg/kg) previously reported. This concentration range has been shown to have biological activity in vitro. If higher concentrations are needed, higher doses could be used, assuming that the pharmacokinetics is linear and that the doses are within the non-toxic dose range. This data can also serve as a guide for planning of a large scale of preclinical pharmacology, in vivo efficacy, and toxicology studies of synthetic microRNAs, as these microRNAs are rather costly.

Analysis of Example

Described herein is a fluorescence hybridization-ligation ELISA method for determination of miRs in a sample. Non-limiting examples of samples include plasma, bone marrow, and cell lysates, such as leukemia cell lysates.

The method provides a quantification method for exogenous microRNA and is ultra-sensitive (having an LLOQ of 5 pM with acceptable precision and accuracy). For example, the pharmacokinetics showed attainable plasma and bone marrow concentrations and terminal half-life comparable to those for currently clinically used anti-sense drugs.

The assay is useful with different capture and detection probes, and can be applicable in quantification of exogenous synthetic miRs and antagomiRs in a variety of biological matrices.

The method is a valuable tool for pharmacokinetics and pharmacodynamics study to guide development of therapeutic agents.

Examples of Uses

As used herein interchangeably, a “miR,” “microRNA,” “miRs,” or “miRNA” refers to the unprocessed or processed RNA transcript from a miR gene. As the miRs are not translated into protein, the term “miRs” does not include proteins. The unprocessed miR gene transcript is also called a “miR precursor,” and typically comprises an RNA transcript of about 70-100 nucleotides in length. The miR precursor can be processed by digestion with an RNAse (for example, Dicer, Argonaut, or RNAse III, e.g., E. coli RNAse III) into an active 19-25 nucleotide RNA molecule. This active 19-25 nucleotide RNA molecule is also called the “processed” miR gene transcript or “mature” miRNA. It is to be understood that the term “miR” as used herein can include one or more of miR-oligonucleotides, including mature miRs, pre-miRs, pri-miRs, or a miR seed sequence. In certain embodiments, a mixture of various miR nucleic acids can also be used. Also, in certain embodiments, the miRs may be modified to enhance delivery.

The miRNA (miR) information is available from the Sanger Institute, which maintains a registry of miRNA at http:/microrna.sanger.ac.uk/sequences/. The miRBase Sequence database includes the nucleotide sequences and annotations of published miRNA from a variety of sources. The miRBase Registry provides unique names for novel miRNA genes that comply with conventional naming nomenclature for new miRNA prior to publication. Also, the miRBase Targets is a resource for predicated miRNA targets in animals.

The active 19-25 nucleotide RNA molecule can be obtained from the miR precursor through natural processing routes (e.g., using intact cells or cell lysates) or by synthetic processing routes (e.g., using isolated processing enzymes, such as isolated Dicer, Argonaut, or RNAase III). It is understood that the active 19-25 nucleotide RNA molecule can also be produced directly by biological or chemical synthesis, without having been processed from the miR precursor.

As used herein, an “isolated” or “synthetic” miR is one which is synthesized, or altered or removed from the natural state through human intervention. For example, a synthetic miR, or a miR partially or completely separated from the coexisting materials of its natural state, is considered to be “isolated.” An isolated miR can exist in substantially-purified form, or can exist in a cell into which the miR has been delivered. Thus, a miR which is deliberately delivered to, or expressed in, a cell is considered an “isolated” miR. A miR produced inside a cell from a miR precursor molecule is also considered to be “isolated” molecule.

Isolated miRs can be obtained using a number of standard techniques. For example, the miRs can be chemically synthesized or recombinantly produced using methods known in the art. In one embodiment, miRs are chemically synthesized using appropriately protected ribonucleoside phosphoramidites and a conventional DNA/RNA synthesizer. Commercial suppliers of synthetic RNA molecules or synthesis reagents include, e.g., Proligo (Hamburg, Germany), Dharmacon Research (Lafayette, Colo., U.S.A.), Pierce Chemical (part of Perbio Science, Rockford, Ill., U.S.A.), Glen Research (Sterling, Va., U.S.A.), ChemGenes (Ashland, Mass., U.S.A.) and Cruachem (Glasgow, UK).

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof. Therefore, it is intended that the invention not be limited to the particular embodiment disclosed herein contemplated for carrying out this invention, but that the invention will include all embodiments falling within the scope of the claims.

### REFERENCES

The publication and other material used herein to illuminate the invention or provide additional details respecting the practice of the invention, are incorporated be reference herein, and for convenience are provided in the following bibliography.

Citation of the any of the documents recited herein is not intended as an admission that any of the foregoing is pertinent prior art. All statements as to the date or representation as to the contents of these documents is based on the information available to the applicant and does not constitute any admission as to the correctness of the dates or contents of these documents.
1. Bartel, D. P. (2004) MicroRNAs: genomics, biogenesis, mechanism, and function. Cell, 116, 281-297.
2. Ambros, V. (2004) The functions of animal microRNAs. Nature, 431, 350-355.
3. Garzon, R., Fabbri, M., Cimmino, A., Cann, G. A. and Croce, C. M. (2006) MicroRNA expression and function in cancer. Trends in molecular medicine, 12, 580-587.
4. He, L., Thomson, J. M., Hemann, M. T., Hernando-Monge, E., Mu, D., Goodson, S., Powers, S., Cordon-Cardo, C., Lowe, S. W., Hannon, G. J. et al. (2005) A microRNA polycistron as a potential human oncogene. Nature, 435, 828-833.
5. Volinia, S., Calin, G. A., Liu, C. G., Ambs, S., Cimmino, A., Petrocca, F., Visone, R., Iorio, M., Roldo, C., Ferracin, M. et al. (2006) A microRNA expression signature of human solid tumors defines cancer gene targets. Proceedings of the National Academy of Sciences of the United States of America, 103, 2257-2261.
6. Calin, G. A., Dumitru, C. D., Shimizu, M., Bichi, R., Zupo, S., Noch, E., Aldler, H., Rattan, S., Keating, M., Rai, K. et al. (2002) Frequent deletions and down-regulation of micro-RNA genes miR15 and miR16 at 13q14 in chronic lymphocytic leukemia. Proceedings of the National Academy of Sciences of the United States of America, 99, 15524-15529.
7. Yanaihara, N., Caplen, N., Bowman, E., Seike, M., Kumamoto, K., Yi, M., Stephens, R. M., Okamoto, A., Yokota, J., Tanaka, T. et al. (2006) Unique microRNA molecular profiles in lung cancer diagnosis and prognosis. Cancer cell, 9, 189-198.
8. Calin, G. A., Ferracin, M., Cimmino, A., Di Leva, G., Shimizu, M., Wojcik, S. E., Iorio, M. V., Visone, R., Sever, N. I., Fabbri, M. et al. (2005) A MicroRNA signature associated with prognosis and progression in chronic lymphocytic leukemia. The New England journal of medicine, 353, 1793-1801.
9. Pekarsky, Y., Santanam, U., Cimmino, A., Palamarchuk, A., Efanov, A., Maximov, V., Volinia, S., Alder, H., Liu, C. G., Rassenti, L. et al. (2006) Tcl1 expression in chronic lymphocytic leukemia is regulated by miR-29 and miR-181. Cancer research, 66, 11590-11593.
10. Fabbri, M., Garzon, R., Cimmino, A., Liu, Z., Zanesi, N., Callegari, E., Liu, S., Alder, H., Costinean, S., Fernandez-Cymering, C. et al. (2007) MicroRNA-29 family reverts aberrant methylation in lung cancer by targeting DNA methyltransferases 3A and 3B. Proceedings of the National Academy of Sciences of the United States of America, 104, 15805-15810.
11. Meng, F., Henson, R., Wehbe-Janek, H., Ghoshal, K., Jacob, S. T. and Patel, T. (2007) MicroRNA-21 regulates expression of the PTEN tumor suppressor gene in human hepatocellular cancer. Gastroenterology, 133, 647-658.
12. Stenvang, J. and Kauppinen, S. (2008) MicroRNAs as targets for antisense-based therapeutics. Expert opinion on biological therapy, 8, 59-81.
13. Cimmino, A., Calin, G. A., Fabbri, M., Iorio, M. V., Ferracin, M., Shimizu, M., Wojcik, S. E., Aqeilan, R. I., Zupo, S., Dono, M. et al. (2005) miR-15 and miR-16 induce apoptosis by targeting BCL2. Proceedings of the National Academy of Sciences of the United States of America, 102, 13944-13949.
14. Mott, J. L., Kobayashi, S., Bronk, S. F. and Gores, G. J. (2007) mir-29 regulates Mc1-1 protein expression and apoptosis. Oncogene, 26, 6133-6140.
15. Barad, O., Meiri, E., Avniel, A., Aharonov, R., Barzilai, A., Bentwich, I., Einav, U., Gilad, S., Hurban, P., Karov, Y. et al. (2004) MicroRNA expression detected by oligonucleotide microarrays: system establishment and expression profiling in human tissues. Genome Res, 14, 2486-2494.
16. Miska, E. A., Alvarez-Saavedra, E., Townsend, M., Yoshii, A., Sestan, N., Rakic, P., Constantine-Paton, M. and Horvitz, H.R. (2004) Microarray analysis of microRNA expression in the developing mammalian brain. Genome Biol, 5, R68.
17. Nelson, P. T., Baldwin, D. A., Scearce, L. M., Oberholtzer, J. C., Tobias, J. W. and Mourelatos, Z. (2004) Microarray-based, high-throughput gene expression profiling of microRNAs. Nat Methods, 1, 155-161.
18. Sempere, L. F., Freemantle, S., Pitha-Rowe, I., Moss, E., Dmitrovsky, E. and Ambros, V. (2004) Expression profiling of mammalian microRNAs uncovers a subset of brain-expressed microRNAs with possible roles in murine and human neuronal differentiation. Genome Biol, 5, R13.
19. Thomson, J. M., Parker, J., Perou, C. M. and Hammond, S. M. (2004) A custom microarray platform for analysis of microRNA gene expression. Nat Methods, 1, 47-53.
20. Baskerville, S, and Bartel, D. P. (2005) Microarray profiling of microRNAs reveals frequent coexpression with neighboring miRNAs and host genes. Rna, 11, 241-247.
21. Chen, C., Ridzon, D. A., Broomer, A. J., Zhou, Z., Lee, D. H., Nguyen, J. T., Barbisin, M., Xu, N. L., Mahuvakar, V. R., Andersen, M. R. et al. (2005) Real-time quantification of microRNAs by stem-loop RT-PCR. Nucleic acids research, 33, e179.
22. Fulci, V., Chiaretti, S., Goldoni, M., Azzalin, G., Carucci, N., Tavolaro, S., Castellano, L., Magrelli, A., Citarella, F., Messina, M. et al. (2007) Quantitative technologies establish a novel microRNA profile of chronic lymphocytic leukemia. Blood, 109, 4944-4951.
23. Wei, X., Dai, G., Marcucci, G., Liu, Z., Hoyt, D., Blum, W. and Chan, K. K. (2006) A specific picomolar hybridization-based ELISA assay for the determination of phosphorothioate oligonucleotides in plasma and cellular matrices. Pharm Res, 23, 1251-1264.
24. Dai, G., Chan, K. K., Liu, S., Hoyt, D., Whitman, S., Klisovic, M., Shen, T., Caligiuri, M. A., Byrd, J., Greyer, M. et al. (2005) Cellular uptake and intracellular levels of the bc1-2 antisense g3139 in cultured cells and treated patients with acute myeloid leukemia. Clin Cancer Res, 11, 2998-3008.
25. Deverre, J. R., Boutet, V., Boquet, D., Ezan, E., Grassi, J. and Grognet, J. M. (1997) A competitive enzyme hybridization assay for plasma determination of phosphodiester and phosphorothioate antisense oligonucleotides. Nucleic acids research, 25, 3584-3589.
26. Mersey, B. D., Jin, P. and Danner, D. J. (2005) Human microRNA (miR29b) expression controls the amount of branched chain alpha-ketoacid dehydrogenase complex in a cell. Hum Mol Genet, 14, 3371-3377.
27. Gilad, S., Meiri, E., Yogev, Y., Benjamin, S., Lebanony, D., Yerushalmi, N., Benjamin, H., Kushnir, M., Cholakh, H., Melamed, N. et al. (2008) Serum microRNAs are promising novel biomarkers. PLoS ONE, 3, e3148.
28. Mitchell, P. S., Parkin, R. K., Kroh, E. M., Fritz, B. R., Wyman, S. K., Pogosova-Agadjanyan, E. L., Peterson, A., Noteboom, J., O'Briant, K. C., Allen, A. et al. (2008) Circulating microRNAs as stable blood-based markers for cancer detection. Proceedings of the National Academy of Sciences of the United States of America, 105, 10513-10518.
29. Liu, C. G., Calin, G. A., Meloon, B., Gamliel, N., Sevignani, C., Ferracin, M., Dumitru, C. D., Shimizu, M., Zupo, S., Dono, M. et al. (2004) An oligonucleotide microchip for genome-wide microRNA profiling in human and mouse tissues. Proceedings of the National Academy of Sciences of the United States of America, 101, 9740-9744.
30. Beuvink, I., Kolb, F. A., Budach, W., Garnier, A., Lange, J., Natt, F., Dengler, U., Hall, J., Filipowicz, W. and Weiler, J. (2007) A novel microarray approach reveals new tissue-specific signatures of known and predicted mammalian microRNAs. Nucleic acids research, 35, e52.
31. Hayashita, Y., Osada, H., Tatematsu, Y., Yamada, H., Yanagisawa, K., Tomida, S., Yatabe, Y., Kawahara, K., Sekido, Y. and Takahashi, T. (2005) A polycistronic microRNA cluster, miR-17-92, is overexpressed in human lung cancers and enhances cell proliferation. Cancer research, 65, 9628-9632.
32. Schmittgen, T. D., Jiang, J., Liu, Q. and Yang, L. (2004) A high-throughput method to monitor the expression of microRNA precursors. Nucleic acids research, 32, e43.
33. Boggs, R. M., Moody, J. A., Long, C. R., Tsai, K. L. and Murphy, K. E. (2007) Identification, amplification and characterization of miR-17-92 from canine tissue. Gene, 404, 25-30.
34. Yu, R. Z., Baker, B., Chappell, A., Geary, R. S., Cheung, E. and Levin, A. A. (2002) Development of an ultrasensitive noncompetitive hybridization-ligation enzyme-linked immunosorbent assay for the determination of phosphorothioate oligodeoxynucleotide in plasma. Analytical biochemistry, 304, 19-25.
35. Efler, S. M., Zhang, L., Noll, B. O., Uhlmann, E. and Davis, H. L. (2005) Quantification of oligodeoxynucleotides in human plasma with a novel hybridization assay offers greatly enhanced sensitivity over capillary gel electrophoresis. Oligonucleotides, 15, 119-131.
36. Brown-Augsburger, P., Yue, X. M., Lockridge, J. A., McSwiggen, J. A., Kamboj, D. and Hillgren, K. M. (2004) Development and validation of a sensitive, specific, and rapid hybridization-ELISA assay for determination of concentrations of a ribozyme in biological matrices. Journal of pharmaceutical and biomedical analysis, 34, 129-139.
37. de Serres, M., McNulty, M. J., Christensen, L., Zon, G. and Findlay, J. W. (1996) Development of a novel scintillation proximity competitive hybridization assay for the determination of phosphorothioate antisense oligonucleotide plasma concentrations in a toxicokinetic study. Analytical biochemistry, 233, 228-233.
38. Corsten, M. F., Miranda, R., Kasmieh, R., Krichevsky, A. M., Weissleder, R. and Shah, K. (2007) MicroRNA-21 knockdown disrupts glioma growth in vivo and displays synergistic cytotoxicity with neural precursor cell delivered S-TRAIL in human gliomas. Cancer research, 67, 8994-9000.
39. Chan, J. A., Krichevsky, A. M. and Kosik, K. S. (2005) MicroRNA-21 is an antiapoptotic factor in human glioblastoma cells. Cancer research, 65, 6029-6033.
40. Krutzfeldt, J., Kuwajima, S., Braich, R., Rajeev, K. G., Pena, J., Tuschl, T., Manoharan, M. and Stoffel, M. (2007) Specificity, duplex degradation and subcellular localization of antagomirs. Nucleic acids research, 35, 2885-2892.
41. Esau, C., Davis, S., Murray, S. F., Yu, X. X., Pandey, S. K., Pear, M., Watts, L., Booten, S. L., Graham, M., McKay, R. et al. (2006) miR-122 regulation of lipid metabolism revealed by in vivo antisense targeting. Cell metabolism, 3, 87-98.
42. Saxena, A., Viswanathan, S., Moshynska, O., Tandon, P., Sankaran, K. and Sheridan, D. P. (2004) Mc1-1 and Bc1-2/Bax ratio are associated with treatment response but not with Rai stage in B-cell chronic lymphocytic leukemia. Am J Hematol, 75, 22-33.
43. Kaufmann, S. H., Karp, J. E., Svingen, P. A., Krajewski, S., Burke, P. J., Gore, S. D. and Reed, J. C. (1998) Elevated expression of the apoptotic regulator Mc1-1 at the time of leukemic relapse. Blood, 91, 991-1000.
44. Galm, O., Herman, J. G. and Baylin, S. B. (2006) The fundamental role of epigenetics in hematopoietic malignancies. Blood Rev, 20, 1-13.
45. Mizuno, S., Chijiwa, T., Okamura, T., Akashi, K., Fukumaki, Y., Niho, Y. and Sasaki, H. (2001) Expression of DNA methyltransferases DNMT1, 3A, and 3B in normal hematopoiesis and in acute and chronic myelogenous leukemia. Blood, 97, 1172-1179.
46. Blum, W. and Marcucci, G. (2005) Targeting epigenetic changes in acute myeloid leukemia. Clin Adv Hematol Oncol, 3, 855-865, 882.
47. Dai, G., Wei, X., Liu, Z., Liu, S., Marcucci, G. and Chan, K. K. (2005) Characterization and quantification of Bc1-2 antisense G3139 and metabolites in plasma and urine by ion-pair reversed phase HPLC coupled with electrospray ion-trap mass spectrometry. Journal of chromatography, 825, 201-213.
48. Wei, X., Dai, G., Liu, Z., Cheng, H., Xie, Z., Marcucci, G. and Chan, K. K. (2006) Metabolism of GTI-2040, a phosphorothioate oligonucleotide antisense, using ion-pair reversed phase high performance liquid chromatography (HPLC) coupled with electrospray ion-trap mass spectrometry. The AAPS journal, 8, E743-755.
49. Shah, V. P., Midha, K. K., Doghe, S., McGilveray, I. J., Skelly, S. P., Yacobi, A., Layloff, T., Viswanathan, C. T., Cook, D. E., McDowall, R. D., Pittman, K. A., and Spector, S. (1992) Analytical Methods Validation: Bioavailability, Bioequivalence, and Pharmacokinetic Studies. Journal of Pharmaceutical Sciences, 81, 4.
50. Krutzfeldt, J., Rajewsky, N., Braich, R., Rajeev, K. G., Tuschl, T., Manoharan, M. and Stoffel, M. (2005) Silencing of microRNAs in vivo with ‘antagomirs’. Nature, 438, 685-689.

1.-44. (canceled)

45. An isolated nucleic acid compound consisting of 3′-GTG ATC AAT-5′ (SEQ ID NO: 12).

46. A compound of claim 45, which further consists of 19 to 25 nucleotides at the 3′ terminus.

47. A compound of claim 45, which further consists of a sense or antisense miR nucleotide sequence at the 3′ terminus.

48. A compound of claim 46, which further comprises biotin at the 3′ terminus.

49. A compound of claim 47, wherein the miR nucleotide sequence is substantially complementary to an artificial miR selected from the group consisting of: 2-MeOPS-miR; exogenous miR; modified miR; synthetic miR; antagomir; SNP of miR; and siRNA.

50. A compound of claim 47, wherein the miR nucleotide sequence is substantially complementary to an artificial miR selected from the group consisting of: artificial miR-29b; artificial miR16-1; and artificial miR-155.

51. An isolated nucleic acid compound consisting of an 8- to 12-base nucleotide 5′ sequence and a sense or antisense miR nucleotide 3′ sequence.

52. An isolated nucleic acid compound consisting of a 9-base nucleotide 5′ sequence and a sense or antisense miR nucleotide 3′ sequence.

53. A probe for detecting a compound of claim 45.

54. A probe of claim 53, which comprises ‘3-ATT GAT CAC-5’ (SEQ ID NO: 7).

55. A probe of claim 53, which further comprises a phosphate at the 5′ terminus

56. A probe of claim 53, which further comprises an immunohistochemical marker at the 3′ terminus.

57. A probe of claim 56, wherein the immunohistochemical marker is a hapten.

58. A probe of claim 57, wherein the hapten is digoxegenin.

59. A kit comprising a compound of claim 45.

60. A kit comprising a compound of claim 53.

61. A kit comprising: a first isolated nucleic acid compound consisting of 3′-19-24 base nucleotide-GTG ATC AAT-5′ (SEQ ID NO: 12); and a second isolated nucleic acid compound consisting of an immunohistochemically-labeled probe for detecting the first isolated nucleic acid compound.

62. A kit of claim 61, which further comprises an antibody selective for the immunohistochemically-labeled probe.

63. A kit of claim 62, which further comprises means to identify the immumochemically-labeled probe.

64. A kit of claim 61, wherein the means to identify comprises Attophos.

65. A method for determining the qualitative or quantitative status of a putative nucleic acid in a test sample, comprising:
a. introducing a compound of claim 46 to a test sample so as to create a first mixture, wherein the 19-25 base nucleic sequence of the compound of claim 46 is substantially complementary to the putative nucleic acid sequence;
b. subjecting the first mixture to hybridizing conditions of approximately 36° to approximately 38° Celsius for approximately 2 hours to approximately 3 hours;
c. introducing a probe for detecting the compound of claim 46 to the first mixture so as to create a second mixture;
d. subjecting the second mixture to ligation conditions;
e. subjecting the second mixture to nuclease digestion conditions of approximately 40 U to approximately 80 U of nuclease at 36° to approximately 38° Celsius for approximately 1.5 hours to approximately 2.5 hours; and
f. identifying the status of putative nucleic acid in the test sample.

66. A method of claim 65, wherein the compound of claim 46 comprises biotin at the 3′ terminus.

67. A method of claim 66, which further comprises a step after step b.) of introducing the second mixture to a biotin-binding substrate.

68. A method of claim 65, which comprises at least one wash step.

69. A method of claim 65, wherein the putative nucleic acid is that it is from an exogenous source and the test sample is a biological sample.

70. A method of claim 69, wherein the biological sample is a human biological sample.

71. A method of claim 65, wherein the putative nucleic acid is a miR.

72. A method of claim 71, wherein the miR is selected from the group consisting of: 2-MeOPS-miR; exogenous miR; modified miR; synthetic miR; antagomir; SNP of miR; and siRNA

73. A method of claim 71, wherein the probe comprises ‘3-ATT GAT CAC-5’ (SEQ ID NO: 7) and an immunohistochemical marker at the 3′ terminus.

74. A method of claim 73, wherein the probe comprises a hapten.

75. A method of claim 74, wherein the hapten is digoxegenin.

76. A method of claim 75, which further comprises the use of an antibody.

77. A method of claim 76, which further comprises the use of Attophos.

78. A method of claim 77, wherein step f.) includes directing an Attophos-binding antibody to a digoxigenin-labeled probe for detecting the compound of claim 46, so as to create a fourth mixture; subjecting the fourth mixture to Attophos so as to create a fifth mixture; subjecting the fifth mixture to phosphatase; and measuring fluorescence of the fifth mixture.

79. A method for assessing pharmacologically specific effects of one or more synthetic microRNAs, comprising the step of: using the method of claim 65.

80. A method for characterizing intracellular pharmacokinetics and/or and pharmacodynamics data of a modified microRNA and its preclinical pharmacokinetics, comprising the step of: using the method of claim 65.

81. A method for qualitative and/or quantitative determination of an analyte in a test sample, comprising the step of: using the method of claim 65.

82. A method for detecting a microRNA at a sensitivity of at least about 30 pM, comprising the step of: using the method of claim 65.

83. A method for detecting a microRNA at a sensitivity of at least about 10 pM, comprising the step of: using the method of claim 65.

84. A method for determining one or more of structural confirmation, identification and differentiation with metabolites or other endogenous substances, comprising the step of: using the method of claim 65.

85. A method for providing relative concentrations of species that are measurable in low concentration ranges, comprising the step of: using the method of claim 65.

86. A method for determining the qualitative or quantitative status of a putative synthetic miR in a human biological test sample, comprising:
a. introducing a 3′-biotin-19 to 23-mer-GTG ATC AAT-5′ (SEQ ID NO: 12) capture template to a human biological test sample so as to create a first mixture, wherein the 19 to 23-mer is substantially complementary to the putative synthetic miR in the sample;
b. subjecting the first mixture to hybridizing conditions of approximately 37° Celsius for approximately 2.5 hours, so as to create a second mixture;
c. introducing the second mixture to a biotin-binding substrate;
d. incubating the second mixture for sufficient time and temperature to allow attachment of the biotin-labeled compound to the biotin-binding substrate;
e. subjecting the biotin-binding substrate to sufficient wash cycles to remove unbound biotin-labeled compound;
f. introducing a detection probe consisting of 3′-digoxigenin-ATT GAT CAC-5′-p (SEQ ID NO: 7) so as to create a third mixture;
g. subjecting the third mixture to ligation conditions;
h. subjecting the biotin-binding substrate to sufficient wash cycles to remove un-ligated detection probe;
i. subjecting the third mixture to nuclease digestion conditions of approximately 60 U of nuclease at approximately 37° Celsius for approximately 2 hours;
j. directing an Attophos-binding antibody to the digoxigenin-labeled probe, so as to create a fourth mixture;
k. subjecting the fourth mixture to Attophos so as to create a fifth mixture;
l. subjecting the fifth mixture to phosphatase;
m. measuring fluorescence of the fifth mixture; and
n. determining the qualitative or quantitative status of the putative synthetic miR in the human biological test sample.
